(19) United States
(12) Patent Application Publication
(10) Pub. No.: US 2026/0151357 A1
(43) Pub. Date: Jun. 4, 2026

(54) Compositions And Methods Of Treatment Of Onychomycosis and Onycholysis

(71) Applicant: Hallux Inc., Laguna Hills, CA (US)

(72) Inventors: Robert L. Orr, San Clemente, CA (US); Christopher Ronald Agee, Lake Forest, CA (US); Geert Cauwenbergh, Laguna Hills, CA (US)

(73) Assignee: Hallux Inc., Laguna Hills, CA (US)

(21) Appl. No.: 19/457,055

(22) Filed: Jan. 22, 2026

Related U.S. Application Data
(63) Continuation-in-part of application No. 17/712,742, filed on Apr. 4, 2022, now Pat. No. 12,576,049.
Application No. 17/712,742 is a continuation-in-part of application No. 17/474,656, filed on Sep. 14, 2021, now Pat. No. 11,400,061.
Application No. 17/474,656 is a continuation of application No. PCT/US2020/054710, filed on Oct. 8, 2020.
(60) Provisional application No. 62/912,494, filed on Oct. 8, 2019.
(60) Provisional application No. 63/748,779, filed on Jan. 23, 2025.

(51) Int. Cl. A61K 31/137; A61K 9/00; A61P 31/10

(52) U.S. Cl. CPC A61K 31/137; A61K 9/0012; A61P 31/10

(57) ABSTRACT

Compositions and methods for treatment of onychomycosis and dermatophytoma are presented. In particularly contemplated aspects, high treatment efficacy is achieved in an atraumatic manner. Moreover, due to the high retention of the antifungal agent in the treatment space and advancement of a newly formed healthy nail plate, onycholysis can be reduced or even entirely reversed, thereby preventing recurrence of infection. Still further, contemplated compositions and methods substantially avoid systemic exposure while achieving high local drug concentration in the treatment space.

This application is a continuation-in-part application of allowed US patent application with the Ser. No. 17/712,742, which was filed Apr. 4, 2022, which is a is a continuation-in-part application of allowed US patent application with the Ser. No. 17/474,656, which was filed Sep. 14, 2021, now U.S. Pat. No. 11,400,061, which is a continuation application of PCT/US2020/054710, which was filed Oct. 8, 2020, which claims priority to US Provisional patent application with the Ser. No. 62/912,494, which was filed Oct. 8, 2019, and each of which are incorporated by reference in its entirety. This application further claims priority to our copending US Provisional patent application with the Ser. No. 63/748,779, filed Jan. 23, 2025, which is also incorporated by reference in its entirety.

### FIELD OF THE INVENTION

The field of the invention is compositions and methods of treatment of the subungual space, especially as it relates to treatment of onychomycosis, dermatophytoma, and/or onycholysis.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Onychomycosis, a chronic fungal infection of nails, affects 2-18% of the population worldwide and the incidence increases with diminished immune competence (e.g., due to diabetes or malignancy), age, or trauma to the toe. Onychomycosis accounts for 50% of the nail disorders seen by physicians. The most common form is distal-lateral subungual onychomycosis (DLSO), an infection primarily of the toenail bed. Causal dermatophytes Trichophyton rubrum, Trichophyton mentagrophytes, and to a lesser extent, Epidermophyton floccosum invade the nail bed from the distal free edge of the nail plate where the hyponychial seal has broken due to disease, trauma, or age. The invasive fungi produce enzymes with keratolytic, proteolytic and lipolytic activities that metabolize keratin and subsequently colonize the nail bed while advancing proximally along its longitudinal ridges and into the ventral nail plate. Mild inflammation ensues, resulting in focal parakeratosis and subungual hyperkeratosis, with the consequence of onycholysis (separation of the nail plate from the nail bed) and thickening of the subungual region. Unfortunately, toenail onychomycosis has long been difficult to treat because many factors affect the treatment outcome: aging patients, long-standing infection, slow growth of thickened nails, co-morbidities, limitations of the treatment (e.g., treatment length, side effects, toxicity, physical limitations of the patient) and presence of fungal masses (dermatophytoma). Hesitation in using oral medications for a non-life-threatening infection exists due to perceived risk of hepatic toxicity and other systemic side effects. Hence, physicians continue to prescribe topical drugs that are associated with limited efficacy and poor patient compliance to a required daily treatment regimen that can last a year or more.

For example, US 2024/0024258 describes topical administration of an antifungal allylamine in a non-aqueous solvent system containing a high concentration of urea, which requires a loading phase and a relatively long maintenance phase, and which may irritate underlying healthy tissue. Alternatively, an antifungal compound may also be administered in a lipophilic formulation as presented in U.S. Pat. No. 12,433,856 where low concentrations of antifungal agents are dispersed in a petrolatum carrier. While significantly less disturbing to healthy tissue, the delivered quantities of the antifungal agent are very low and likely not sufficient to result in significant cure rates. In further known approaches, an antifungal can be delivered from a drug eluting patch using thiourea as a penetration enhancer as described in U.S. Pat. No. 12,303,491. While convenient, such method tends to use lower concentrations of antifungal agent that will once more not produce significant cure rates in many cases.

Other topical treatments include those as shown in U.S. Pat. No. 9,302,009, where a formulation having a surface tension of 40 dynes/cm or less, and not forming a solid film when applied to the surface of a nail, provides enhanced penetration of the antifungal agent through an intact nail and into the nailbed. In yet another approach, as disclosed in US 2011/0066134, antifungal agents are delivered through the nail plate via iontophoresis. However, such method requires dedicated equipment and is difficult to adhere over prolonged periods.

In further known examples, US 2013/01222097 and U.S. Pat. No. 8,354,095 teach placement of antifungal micro-implants into the hyponychium, and intradermal placement into the tissue surrounding the nail plate, as well as into tissue between the nail plate and the bone of the phalanx. As will be readily apparent, such treatment will be painful and will further risk injury to the nailbed. In a similar manner, US 2022/0354783 discloses subcutaneous injection of solid biodegradable sustained-release polymers, which may be painful and requires diffusion of the active agent to the site of infection.

To avoid difficulties associated with topical administration or intradermal injection, U.S. Pat. No. 8,404,751 discloses various compositions and methods where a semi-solid carrier containing an antifungal agent is forced under the nail or between the nail and the nail bed using a cannula or hypodermic needle. Similarly, U.S. Pat. No. 9,498,612 discloses the creation of a channel or space in the subungual space by insertion of a cannula to so allow placement of a drug-eluting implant Unfortunately, such subungual administration is traumatic and tends to lead to onycholysis, the very condition that facilitates onychomycosis. Moreover, the antifungal effect in such methods is often less than desirable.

Thus, even though various systems and methods of treating onychomycosis are known in the art, all or almost all of them suffer from several drawbacks. Therefore, there remains a need for improved and atraumatic systems and methods for effectively treating onychomycosis with minimal systemic exposure while achieving high concentrations of an antifungal agent in the nail bed and plate. There also remains a need for treatment of dermatophytoma fungal masses, which may be protected by biofilm structures, and as a result are highly resistant to pharmacologic treatment.

### SUMMARY OF THE INVENTION

The inventive subject matter is directed to various high concentration antifungal compositions and methods of atraumatic and effective subungual treatment of onychomycosis and dermatophytoma, which as a consequence also reduces fungal induced onycholysis without exposing the patient to systemic toxicity over the duration it takes to re-grow a disease-free nail.

In one aspect of the inventive subject matter, the inventors contemplate a method of treating dermatophytoma that includes a step of subungually and atraumatically administering a composition proximal to and/or into a polysaccharide matrix of a dermatophytoma fungal mass located in a subungual space. In especially contemplated methods, the composition has a viscosity of between about 500-2,500 cP (mPa*s) and comprises an antifungal agent in a hydrophobic solvent, and it is generally contemplated that the antifungal agent has at a concentration that is effective to inhibit growth of a fungal pathogen in the subungual space and to inhibit growth of the fungal pathogen in the nail plate as ascertained by a negative culture of distal nail bed tissue and nail clippings. Most typically, the composition is administered repeatedly to inhibit further growth of the dermatophytoma mass and in time fully eradicate the encapsulated causative fungal pathogens.

In some embodiments, the hydrophobic solvent is selected isostearic acid, benzyl alcohol, diisopropyl adipate, diethyl sebacate, and/or isopropyl myristate, and the composition may further include the hydrophilic solvent and/or the polymeric film forming agent. Most typically the antifungal agent is an allylamine, and preferably terbinafine free base. Additionally, it is contemplated that the antifungal agent is present at a concentration that sustains an antifungal drug concentration of at least 1× minimum inhibitory concentration (MIC) for at least 30 days. Moreover, the composition also preferably is formulated to prevent systemic absorption of the antifungal agent substantially completely into the blood circulation of a subject. While not limiting to the inventive subject matter, the composition is typically administered monthly for several months, and even more typically for at least one year.

In another aspect of the inventive subject matter, the inventors contemplate a method of treating onychomycosis that includes a step of subungually and atraumatically administering a composition into a subungual space at an asymmetric schedule. Preferably, the composition includes a pharmaceutically acceptable carrier comprising an antifungal agent in a hydrophobic solvent, an optional hydrophilic solvent, an optional polymeric film forming agent, and the composition has a viscosity of between about 500-2,500 cP (mPa*s). It is further generally preferred that the asymmetric schedule comprises a loading schedule that extends over at least two weeks (e.g., four weeks) and a maintenance schedule that extends over at least six month (e.g., 11 months). In such methods, it is generally preferred that the loading schedule delivers, normalized per month, at least twice the amount of the antifungal agent to the subungual space relative to the maintenance schedule.

In some embodiments, the hydrophobic solvent is isostearic acid, benzyl alcohol, diisopropyl adipate, diethyl sebacate, and/or isopropyl myristate, in at least some embodiments the composition may further comprise the hydrophilic solvent and/or the polymeric film forming agent. Typically, but not necessarily, the antifungal agent is terbinafine free base at a concentration of at least 10 wt % (e.g., about 20 wt %, or about 40 wt %). It is further contemplated that the asymmetric schedule may extend over one year. For example, suitable loading schedules may extend over one month and comprise one initial and two biweekly administrations, while suitable maintenance schedules may extend over about one year and comprise eleven once-monthly administrations.

Using such methods, the Investigator Global Assessment (IGA) disease severity grading scale may improve at least 2 grades after completion of the asymmetric schedule. Alternatively, or additionally, the asymmetric schedule may, upon completion, produce clinical cure (i.e., 0% disease involved nail area) and/or mycological cure (i.e., negative KOH and culture).

In still another aspect of the inventive subject matter, the inventors also contemplate a method of treating onychomycosis that includes a step of subungually and atraumatically administering a composition into a subungual space at a metronomic schedule, wherein the composition comprises a pharmaceutically acceptable carrier comprising an antifungal agent in a hydrophobic solvent, an optional hydrophilic solvent, an optional polymeric film forming agent, and wherein the composition has a viscosity of between about 500-2,500 cP (mPa*s). Most preferably, the composition is formulated such that, upon completion of the metronomic schedule, (1) growth of the fungal pathogen on the nail bed and/or in the nail plate is inhibited as ascertained by a negative culture of distal nail bed tissue and/or nail clippings, (2) therapeutically effective concentrations of antifungal agent are present in the nail plate, and (3) antifungal agent is substantially absent in systemic circulation.

In some embodiments, the hydrophobic solvent is isostearic acid, benzyl alcohol, diisopropyl adipate, diethyl sebacate, and/or isopropyl myristate, and the composition may further comprise the hydrophilic solvent and/or the polymeric film forming agent. As noted earlier, the antifungal agent may be terbinafine free base, typically at a concentration of at least 10 wt %. In further embodiments of such method, the mean Cmax of the antifungal agent in plasma post administration will be equal or less than 0.13 ng/ml, and/or the mean terbinafine concentration in nail clippings is about 100 μg/mg post administration. While not necessary, it is preferred that the metronomic schedule has a loading schedule and a maintenance schedule.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 depicts a series of photographs taken over time illustrating successful treatment of onychomycosis with complicating dermatophytoma.

FIG. 2 depicts a series of graphs showing various metrics of treatment over time for the treated target toe of FIG. 1.

### DETAILED DESCRIPTION

The inventors have now discovered liquid pharmaceutical compositions for subungual administration that allow for a high concentration of antifungal agent in a pharmaceutically acceptable carrier. Notably, such compositions have proven to be highly effective and allowed for simple and atraumatic placement into the subungual space. Moreover, the compositions and methods presented herein are not only suitable for treatment of onychomycosis but are also effective in the treatment of dermatophytoma, and as a result also effective in reducing fungal induced onycholysis.

In especially contemplated embodiments, typical compositions comprise a carrier that includes a hydrophobic solvent, an optional hydrophilic solvent, and an optional polymeric film forming agent, and the pharmaceutical composition has a viscosity that not only allows for targeted delivery to the subungual space via a small-bore cannula, but also allows for lateral, proximal, and distal passive distribution of the liquid pharmaceutical composition through the subungual space, typically via translation, capillary action, and/or surface tension. Advantageously, and as is shown in more detail below, the compositions presented herein allow for diffusion of the antifungal agent into the nail plate at concentrations substantially at or above the minimum inhibitory concentration (MIC) for the typical pathogenic fungi such as Trichophyton rubrum, Trichophyton mentagrophytes, and to a lesser extent, Epidermophyton floccosum, while substantially completely avoiding systemic absorption into the circulatory system of a patient as is also shown in more detail below.

Advantageously, the compositions presented herein also enable the placement and distribution of the antifungal agent at high concentrations into, proximal to, and through the polysaccharide matrix of a dermatophytoma, and are therefore also suitable for treatment of dermatophytoma. In this context, it should appreciated that a dermatophytoma is typically characterized as a polysaccharide matrix or biofilm structure that encapsulates a mass of fungus, and that in cases where the polysaccharide matrix is extensive, decreased penetration of antifungal agents makes the condition generally resistant to pharmacologic antifungal monotherapy. In heretofore known cases, surgical removal of the nail plate in combination with oral terbinafine was often the only somewhat effective manner of treatment.

As used herein, it is important to recognize that the term “subungual space” refers to a pre-existing void space that is present between the nail bed and the nail plate, and that space enables fungal growth in people with onychomycosis. Viewed from a different perspective, the subungual space may be considered a contiguous open space that may be empty, or that may include debris from keratin, collagen, and/or fungal material (such as a polysaccharide matrix). Therefore, the subungual space is typically a space of variable geometry that may extend over a significant portion of the nail organ, and that may further include areas that are co-extant with current and/or prior fungal growth. Thus, from a drug administration perspective, the subungual space is a space that is accessible from a location outside the nail organ without damage to or separation of the nail plate and nail bed upon access. For example, administration of a composition as contemplated herein can be performed with a small gauge blunt-tip cannula without producing a punctate bleed and onycholysis.

The term “subungual space” is therefore distinct from the term “treatment space” in that the treatment space will also include areas that are outside the subungual space but in which the active ingredient will be present (e.g., via diffusion), preferably in an amount that is at least the MIC (minimum inhibitory concentration) of the active agent. Consequently, the treatment space in most cases will be more extensive than the subungual space and will include an area that has not directly contacted a treatment composition. For example, the treatment space will typically include a space within which the active ingredient is present due to diffusion and/or nail growth. For example, where a highly concentrated terbinafine solution was atraumatically administered into the subungual space, the treatment space will include space adjacent to the subungual space in which the terbinafine is present in at least MIC and a portion of the nail plate into which the terbinafine has diffused. Notably, such region in the nail plate may continually expand the treatment space as the nail grows distally.

With regard to the term “liquid” as used in conjunction with contemplated compositions herein, it is noted that the term liquid refers to compositions that comprise a liquid component (typically a solvent that is liquid at room temperature) that may have one or more other components (e.g., antifungal agent) dissolved, dispersed, or otherwise distributed therein. Additionally, a solid may be de-stabilized into an amorphous phase, which may appear as a liquid. Consequently, the terms “liquid” compositions and “flowable” compositions are used interchangeably herein. Most typically, liquids contemplated herein will have a dynamic viscosity of between 500 and 2,500 cP.

Consequently, it should be appreciated that contemplated compositions and methods allow for therapeutic coverage of the entire mycotic nail bed, eradicating the proliferating and non-proliferating spores quickly so that new disease-free nail plate and nail bed below can replace the mycotic tissue (concurrent with linear growth). Due to the relatively high concentration of active agent (preferably terbinafine), a reservoir is created that can spread from the site of deposition proximally through the ridges and caverns of the nail bed. Moreover, due to the hydrophobic nature of the carrier, the active agent is quickly absorbed into the nail plate and so forms an antifungal barrier that may prevent proximal and ventral fungal invasion. It should also be recognized that the hydrophobic nature of the carrier will reduce systemic absorption of the active agent, while at the same to promote partitioning of the active agent into the nail plate.

In addition, contemplated formulations have a viscosity that balances fluid distribution in the subungual space with the ability to inject the formulation through small-bore cannulas (e.g., blunt-tip 30-gauge cannula) to so prevent or significantly reduce further lifting of the nail plate (onycholysis). Such cannulas are also blunt enough to allow delivery of the active agent into the center of the mycotic nail bed space without trauma or damage to the nail bed epithelium, and precise enough to allow a physician to treat entry points such as the lateral edge, and to allow for delivery of the liquid proximal to or into the tough-to-treat fungal mass (dermatophytoma). Viewed from a different perspective, proper viscosity of contemplated formulations will balance numerous requirements for a therapeutic effect: Retention of a liquid composition in the subungual space at therapeutic quantities, while allowing for administration and passive distribution (e.g., via capillary action and/or mechanical force due to toe movement) of the liquid formulation throughout the subungual space without loss due to leakage.

Typically, contemplated compositions are formulated such that at least 50%, or at least 60%, or at least 70%, or at least 80%, or at least 90%, or at least 95% of the administered volume of the liquid pharmaceutical composition will remain in the subungual space without being drained and/or expelled upon ordinary daily activity. Moreover, film forming agents may promote distribution of the formulation throughout substantially the entire subungual space, typically by capillary action, wicking, and/or surface tension. In still further contemplated aspects, the film forming agent may also promote penetration of the formulation through the polysaccharidic matrix of a dermatophytoma located between a nail bed and a nail plate of a patient diagnosed with onychomycosis. Advantageously, such penetration allows treatment of areas previously deemed untreatable.

Access to the subungual space is typically via manual insertion of a blunt-tip cannula into the pre-existing void space (which may contain debris and other material) of the subungual space by a practitioner. In most embodiments, suitable cannulas are small-bore cannulas, typically at least 25 gauge, more preferably 27 gauge, and even more preferably 30 gauge, or even smaller. Such cannulas may have one or more lateral ports to facilitate delivery of the formulation into the subungual space. Advancement of the cannula (that is attached to the body of a pre-filled syringe) will generally not require any force sufficient to induce trauma, bleeding, and/or partial avulsion. Most typically, between 10 and 200 μL of the liquid pharmaceutical composition will be atraumatically injected into the subungual space. It should also be appreciated that in most embodiments gentle, non-traumatic mechanical piercing action of the blunt-tip cannula can penetrate into and/or through the porous polysaccharide matrix produced by a dermatophytoma. As such, contemplated formulations allow for lateral flow into and through the porous matrix. Moreover, the viscosity of the liquid composition allows for lateral flow within the subungual space, typically without being expressed excessively and lost from the subungual space in significant quantities.

Exemplary formulations suitable for use herein are shown in Table 1. Most notably, very high concentrations of terbinafine could be achieved in a hydrophobic carrier. However, stability at standard conditions (at 25° C. and 60% relative humidity, 9 months storage) was shown only for formulations F7 and F8, whereas formulations F1-F6 ultimately precipitated. Of course, it should be noted that where lower concentrations of the antifungal agent are employed, stability results will be different and as such, formulations F1-F6 are not excluded from use in conjunction with the teachings presented herein. For example, the antifungal agent may be present in an amount of about 10 wt %, or 15 wt %, or 20 wt %, or 25 wt %, or 30 wt %, or 35 wt %. In this context, it should also be noted that the pharmaceutical compositions will be effective as a monotherapy (i.e., will not include a second drug), but that such compositions can include additional therapeutic agents.

TABLE 1

| | | Wt % Composition | | | | | | | | | |
|---|---|---|---|---|---|---|---|---|---|---|---|
| Component | Function | F1 | F2 | F3 | F4 | F5 | F6 | F7 | F8 | F9 | F10 |
| Terbinafine free | Active | 50.00 | 50.00 | 50.00 | 50.00 | 50.00 | 49.40 | 42.00 | 42.00 | 42.00 | 42.00 |
| base | ingredient | | | | | | | | | | |
| Butylated | Anti- | 0.10 | 0.10 | | 0.10 | 0.10 | 0.10 | 0.20 | 0.20 | 0.20 | 0.20 |
| hydroxytoluene | oxidant | | | | | | | | | | |
| Benzyl alcohol | Solvent | 2.50 | 2.50 | 0.10 | 2.50 | 2.50 | 2.50 | 2.70 | 2.70 | | |
| Benzyl benzoate | Solvent | | | 49.90 | | | | | | | |
| Diisopropyl | Co-solvent | | 20.00 | | 20.00 | 20.00 | 20.00 | 20.00 | 20.00 | | |
| adipate | | | | | | | | | | | |
| Diethyl sebacate | Co-solvent | 22.50 | 24.00 | | 24.00 | 24.00 | 24.00 | | | | |
| Isopropyl | Co-solvent | | 3.40 | | | | | 31.10 | 32.10 | 5.80 | 7.80 |
| myristate | | | | | | | | | | | |
| Isostearic acid | Solvent, | 24.90 | | | 3.40 | | | | | 25.00 | 22.00 |
| | Diluent | | | | | | | | | | |
| Isostearyl alcohol | Diluent | | | | | 2.50 | | | | | |
| Dimethyl | Solvent | | | | | | | | | 15.00 | 15.00 |
| isosorbide | | | | | | | | | | | |
| Propylene | Solvent | | | | | | | | | 5.00 | 5.00 |
| carbonate | | | | | | | | | | | |
| D,L-Lactic acid | Solvent | | | | | | | | | 7.00 | 7.00 |
| Ethanol | Gelling | | | | | 0.70 | | | | | |
| | vehicle, | | | | | | | | | | |
| | solvent | | | | | | | | | | |
| Hydroxypropyl | Gel agent, | | | | | 0.20 | | | | | |
| cellulose | viscosity | | | | | | | | | | |
| | increase, | | | | | | | | | | |
| | film former | | | | | | | | | | |
| Ethyl cellulose | Gel agent, | | | | | | 4.00 | 4.00 | 3.00 | | 1.00 |
| | viscosity | | | | | | | | | | |
| | increase, | | | | | | | | | | |
| | film former | | | | | | | | | | |

Due to the generally hydrophobic nature of the liquid formulation, along with the high concentrations of antifungal agent supported by the carrier, it should be appreciated that multiple advantages can be achieved, leading to the desirable results shown herein. Most significantly, and as is shown in more detail below, the hydrophobic nature of the formulation will limit the diffusion of the antifungal agent into the systemic circulation to a significant degree while at the same time supporting concentrations of the antifungal agent that well exceed MIC of the antifungal agent with respect to a subungual pathogen. Additionally, due to the hydrophobic nature of the formulation, diffusion of the antifungal agent into the nail plate is enabled to exceptionally high levels as is also shown in more detail below.

As such, treatment of onychomycosis and dermatophytoma is not just limited to the subungual space to kill and/or inhibit growth of a fungal pathogen but also extends into the nail plate. Thus, as live fungus is eliminated from the subungual space and surrounding nail bed area, any residual fungus will not be able to escape into the nail plate. In contrast, as the nail plate advances distally, a healthy (disease-free) nail plate displaces the previously infected nail plate without being reinfected. Consequently, the nascent nail plate will be attached to the nailbed, and after sufficient time completely replace a diseased nail plate. Viewed from a different perspective, the treatment contemplated herein may also be effective to reduce or even entirely reverse onycholysis as healthy new nail reattaches to the nail bed.

In most cases, subungual administration of the formulations presented herein will be repeated over time to ensure substantially continuous presence of the antifungal agent. Most typically, the antifungal agent will present in the subungual space at a concentration that sustains an antifungal drug concentration of at least 1× minimum inhibitory concentration (MIC) or at least 2×MIC, or at least 5×MIC, or at least 10×MIC for at least 30 days, or at least 45 days, or at least 60 days, and even longer. For example, it is contemplated that formulations presented herein may be administered multiple times over at least six months, or at least nine months, or a year and even longer. Multiple administrations will typically be between once quarterly and once weekly. Of course, it will be readily appreciated that administration may follow a metronomic schedule (i.e., multiple administrations at same time intervals) or an asymmetric schedule (i.e., multiple administrations at different time intervals). For example, a metronomic schedule may be once monthly administration (with possible deviations of no more than four days per administration), while an asymmetric schedule may comprise some initial administrations at a biweekly pace (loading schedule), which is then followed by administrations at regular and longer intervals such as once monthly administration (maintenance schedule). As will be readily appreciated, the frequency of administration in either schedule may be influenced by various factors (e.g., concentration of antifungal agent, type of pathogen, etc.) and as such may be adjusted accordingly to be, for example, every other day, weekly, biweekly, once every three weeks, once monthly, every six weeks, bimonthly, quarterly, etc.

Most typically, and as is discussed in more detail below, upon completion of a treatment regimen with multiple administrations, treatment may result in an improvement of at least 2 grades on the Investigator Global Assessment (IGA) disease severity scale. Therefore, in some aspects, the treatments contemplated herein may result in clinical cure (i.e., 0% disease-involved nail area) and/or mycological cure (i.e., negative KOH and culture). In other aspects, treatment may result in either negative KOH (potassium hydroxide) or a negative culture from distal nail bed tissue or clippings. In addition to the various benefits as presented herein, it should also be noted that several other advantages can be realized with the composition and methods of the inventive subject matter. The methods presented herein provide an ideal alternative for older patients who have limited eyesight or lack the dexterity to reach their lower extremities and apply medicine to their toenails. Moreover, monthly or bimonthly application may provide better adherence than daily “at home” applications, and dermatophytomas can be treated with higher expectations of resolution while leaving the nail intact. Finally, podiatrists or dermatologists can visualize where the drug should be applied, and multiple toes can be treated in one session. Lastly, there is no risk for significant systemic exposure to the drug.

Moreover, and as is shown in more detail below, patients diagnosed with onychomycosis complicated by dermatophytoma can be treated by atraumatic subungual administration of a high-concentration antifungal composition comprising terbinafine free base in a hydrophobic carrier. In this context, it should be noted that patients with visible fungal masses protected by a polysaccharide matrix within the subungual space represent a population that is known to be difficult to treat and that is often resistant to conventional topical and systemic therapies. In contrast, contemplated compositions were administered periodically into the pre-existing subungual space using a blunt-tip micro-cannula, without inducing bleeding, nail plate avulsion, or clinically meaningful trauma. Following treatment over several weeks, most treated dermatophytoma-affected nails demonstrated marked clinical improvement, including reduction or resolution of the fungal mass, decreased disease-involved nail area, and progressive distal outgrowth of clear, healthy nail plate. Mycological evaluation of distal nail clippings collected after completion of the treatment regimen revealed negative fungal cultures in treated subjects, indicating eradication of the causative dermatophyte organisms within the fungal mass. Pharmacokinetic evaluation indicated that systemic exposure to terbinafine was minimal. Plasma concentrations of terbinafine and its primary metabolite were below or near the lower limit of quantification, while therapeutically effective concentrations of terbinafine were detected locally in nail tissue. Beneficially, treatment was well tolerated in this difficult-to-treat population and reported treatment-related effects were localized and transient, with no evidence of clinically meaningful systemic toxicity or safety concerns.

### EXAMPLES

### Controlled Multi-Subject Study

Study protocol: One great toe was selected by investigators at baseline as the target toe to fulfill the primary and secondary efficacy assessment at week 52. The great toe with the greater acceptable disease involvement by nail surface area was proposed at screening and later confirmed at baseline by positive mycology tests (positive KOH and culture) and acceptable nail characteristics and infection criteria. In some cases, both great toes had similar acceptable disease involvement at the screen visit and were sampled for mycology. If both toes were positive, the investigator would select the great toe with the greater disease involvement as the target toe. However, if both toes were also similar in disease area, the investigator would base his/her selection on other subjective treatment-related factors such as the availability of subungual space, the degree of subungual debris and hyperkeratosis, and the location of disease involvement on the nail plate surface with more central and widely spread areas of infection preferred over predominantly lateral edge disease. One trial location was allowed to perform KOH screening on-site using a wet mount method. The other three sites sent their mycology specimens to a central mycology lab for KOH confirmation using calcofluor white stain. The central mycology lab performed the culture for dermatophyte (sabouraud dextrose agar) and final KOH test at week 52 on all patients.

Patients were instructed not to clip, trim, or file treated toes during the study. Investigators trimmed nails at each visit. Pedicures were not allowed on any toe and following treatment patients were instructed not to swim, bathe, shower, or spa for a minimum of eight hours. Beginning at the screen visit and repeated all visits thereafter prior to any in-study activity, patients were asked about their compliance to a foot care regimen of eleven activities (e.g., wearing of clean socks, restrictions on nail polish, use of cosmetics, etc.). Vital signs were evaluated at screen visit and week 52. A lower extremity exam at those visits focused on foot and toe circulation and looked for evidence of tinea pedis.

When visually assessing the extent of onychomycosis on the target great toe each site visit, the investigator observed its clinical signs on an iPad® captured image (high resolution cross polarized photograph) of the nail plate and bed and digitally traced that involved area on the image using an Apple® pen while in the presence of the patient. The marked sections within the nail's perimeter (bound by the proximal, lateral, distal nail folds) were quantified as percent (%) involved area by Canfield Capture Mobile™ (CCM), image analysis software pre-installed on the iPad® for use exclusively in HSG-201. The planimetry and linear length data generated by CCM (i.e. total nail plate area/length, involved nail plate area/length, uninvolved nail plate area/length, all expressed in millimeters) enabled visit-to-visit monitoring of treatment response, and particularly the outward (distal) growth of clear, uninvolved nail over the trial duration. A target toenail assessed as 0% to ≤10% disease-involved area was categorized on the IGA disease severity scale as grade 0 or 1. Complete Cure required an IGA grade 0 at end of study (week 52) accompanied by negative mycology (negative KOH and culture).

At the screen visit, patients were asked about their nail trimming habits in a Foot Care Questionnaire. Thirty-five (35/47, 74.4%) patients acknowledged clipping their great toenail at least once every four weeks. To calculate a 4-week linear growth rate (NGR), investigators (or study coordinators) used a sterile scalpel (#15) at screen to cut a notch or groove in the nail plate surface midline and then measured by digital caliper four weeks later (baseline) the distal outgrowth of this notch in millimeters. Canfield (CCM) imaging confirmed this growth rate by comparing uploaded screen and baseline images of a digital marker placed over the physical notch and the outward distance of this marker over the same time period. Enrollment required a NGR ≥1 mm/month (e.g. 0.25 mm/week adjusted for four weeks). Sixteen patients (16/47, 34%) had NGR <1 mm calculated by Canfield despite their admission into the study by the trial sites based on caliper readings.

Using a high-resolution balance scale (e.g., Mettler Toledo™), study coordinators weighed each pre-filled syringe before and after treatment to a toe (syringe only—no cap or attached cannula). After conversion, applying the density of HSG 42 (approximately 0.93), the estimated expelled volume corresponding to 1) the target toe, 2) each smaller toe treated on that foot (target foot), 3) each toe treated on the other foot (non-target foot) was calculated as follows:

$$\begin{matrix}Estimated expelled volume = ( ( Pre - dose Total Weight ) - ( Post - dose Total Weight ) ) / ( Density of HSG 42 ) \\ Total Weight = Weight of syringe applicator + Weight if micro - cannula and cover\end{matrix}$$

Utilizing subungual space, formulation of F8 in Table 1 was administered onto the nail bed of the target toe, and in the same manner to other clinically diagnosed toes of the target and non-target feet on eight dose occasions (treatments), with treatments scheduled week 0, week 4, week 8, week 12, week 20, week 28, week 36, and week 44. Aided sometimes by a 2 mm curette used as probe, the investigator determined how many cannulations the target toe would accommodate (up to three allowed per protocol). After a dose administration, the investigator rubbed by gloved index finger any excess drainage (spillover) from the nail bed evenly onto adjacent periungual skin (hyponychium, distal free edge of toenail, proximal and distal fold). The patient then lifted their treated toe vertically for sixty seconds so the investigator could apply light pressure to the toenail with thumb and induce further translation of HSG 42 within the subungual space.

Immediately following each treatment, on the third day after each treatment, and again several days ahead of the next scheduled treatment visit, the investigator or study coordinator would communicate with the patient to discuss any incidence of site reactions and/or pain (i.e., Treatment Assessment Scale, Pain Intensity Scale entries) that may require immediate attention by the medical monitor or investigator, and for recording in the patient's CRF.

After the last treatment visit (week 44), patients returned for two non-treatment visits at week 48 and week 52 (end of study). Final mycology samples were collected at week 52. Treated patients were assessed for safety at all visits and for efficacy at week 52.

Safety variables over the study period included reported adverse events, treatment site reactions (including surrounding periungual skin at dose cannulation entry points (i.e. hyponychium and proximal, lateral and distal nail folds), pain, clinically significant changes in medical condition and medication use, plasma pharmacokinetics, and clinical laboratory evaluation of liver function, serum chemistry, urinalysis, and complete blood count (Section 9.5).

The pharmacokinetic (PK) assessments were undertaken to characterize concentrations of terbinafine and major metabolite N-desmethyl terbinafine present in patient plasma and in distal toenail clippings of the target toe by a validated LC/MS/MS method (qualified method in the case of distal toenail). The lower limit of quantification (LLOQ) for the plasma bioanalytical method is 0.04 ng/mL for parent and metabolite, and 0.5 ng/ml for the nail bioanalytical method.

HSG 42 was topically applied first to the target toe. Smaller toes of the target foot (i.e., target great toe and smaller toes of same foot) were then treated with the same pre-filled syringe but fitted with a new micro-cannula for each small toe on that target foot. As a result of the limited volume available in subungual space to treat the nail bed of great and smaller toes, the Sponsor estimated a maximum dose of no more than 100 μL (39 mg) would be administered to all toes combined of the same foot each treatment visit (200 μL (78 mg) if toes of both feet were treated). Treatments were scheduled Week 0, 4, 8, 12, 20, 28, 36 and 44, for a total of eight dose occasions over the 52-week study period.

Sample collection procedures, proper specimen ID, use of aliquot tubes, storage, and shipment procedures for the assay of terbinafine and metabolite concentrations in plasma and distal nail clippings were outlined in the pharmacokinetics (PK) plan and lab manual provided to the study sites by KCAS Bioanalytical and Biomarker Services.

Characterization of human plasma concentrations of terbinafine and N-desmethyl terbinafine (ng/mL) over the study period was determined by KCAS using a validated LC/MS/MS method. The lower limit of quantification (LLOQ) for the bioanalytical method is 0.04 ng/ml for both parent and metabolite. Study site plasma collections from male and female patients in the PK subpopulation were scheduled at baseline (pre-dose day 1), day 4, and pre-dose day 29 (week 4) day 57 (week 8), day 85 (week 12).

Characterization of concentrations of terbinafine and N-desmethyl terbinafine in distal nail clippings (ug/g) was determined by KCAS using a qualified LC/MS/MS method. The low-curve method was linear over the range of 0.500 to 64.0 ng/ml with a 0.100 mL aliquot volume for both terbinafine and N-DT. The high curve method was linear over the range of 50.0 to 100,000 ng/ml with a 0.100 mL aliquot volume for both terbinafine and N-DT. Nail specimens from the target toe of all patients were collected pre-treatment at baseline (day 1), week 4 (day 29), week 8 (day 57), week 12 (day 85), week 20 (day 141), week 28 (day 197), week 36 (day 253), week 44 (day 309), week 48 (day 337), and week 52 (day 365).

Each patient in the plasma PK analysis set who completed the PK portion of the study without any protocol deviations that would likely affect results were included in the plasma PK study report (analysis population). All patients participating in the plasma PK portion of the study but excluded from the PK analysis population were described in the PK report.

Values below the LLOQ were to be referred to as below the quantification limit (BQL). BQL values are imputed as 0 for all calculations including non-compartmental analysis. Missing pre-dose PK samples concentrations are imputed as 0 for analysis. All other missing PK sample concentrations were imputed for the non-compartmental analysis.

Table 2 shows the treatment procedure by visit.

TABLE 2

| Visit | | | V02 | V03 | V04 | V05 | V06 | V07 | V08 | V09 | Total |
|---|---|---|---|---|---|---|---|---|---|---|---|
| # Patients/target toes | | n | 47 | 46 | 46 | 46 | 46 | 46 | 45 | 45 | 367 |
| Total Cannulations | | N | 138 | 136 | 135 | 138 | 137 | 136 | 131 | 129 | 1080 |
| (3 allowed/ treatment) | | | | | | | | | | | |
| # of Cannulations | | Mean | 2.9 | 3.0 | 2.9 | 3.0 | 3.0 | 3.0 | 2.9 | 2.9 | — |
| Ease of cannulation | | | | | | | | | | | |
| Very easy | | n (%) | 54 | 51 | 51 | 54 | 54 | 54 | 50 | 44 | 412 |
| | | | (39.1) | (37.5) | (37.8) | (39.1) | (39.4) | (39.7) | (38.2) | (34.1) | (38.1) |
| Easy | | n (%) | 79 | 82 | 80 | 84 | 80 | 82 | 78 | 82 | 647 |
| | | | (57.2) | (60.3) | (59.3) | (60.9) | (58.4) | (60.3) | (59.5) | (63.6) | (59.9) |
| Difficult | | n (%) | 5 | 3 | 4 | 0 | 3 | 0 | 3 | 3 | 21 |
| | | | (3.6) | (2.2) | (3.0) | (0) | (2.2) | (0) | (2.3) | (2.3) | (1.9) |
| Depth of cannulation | | Median | 3 | 3 | 3 | 3 | 3 | 3 | 3 | 3 | — |
| ≤1.5 | mm | n (%) | 0 | 1 | 4 | 4 | 4 | 8 | 10 | 4 | 35 |
| | | | (0) | (1) | (3) | (3) | (3) | (6) | (8) | (3) | (3) |
| <3 | mm | n (%) | 4 | 8 | 12 | 7 | 4 | 6 | 11 | 4 | 56 |
| | | | (3) | (6) | (9) | (5) | (3) | (4) | (8) | (3) | (5) |
| 3 | mm | n (%) | 82 | 72 | 67 | 84 | 82 | 66 | 70 | 83 | 606 |
| | | | (59) | (53) | (50) | (61) | (60) | (49) | (53) | (64) | (56) |
| 3-5 | mm | n (%) | 31 | 31 | 22 | 20 | 24 | 22 | 17 | 8 | 175 |
| | | | (22) | (23) | (16) | (14) | (18) | (16) | (13) | (6) | (16) |
| 3 and 3-5 | mm | n (%) | 113 | 103 | 89 | 104 | 106 | 88 | 87 | 91 | 781 |
| | | | (82) | (76) | (66) | (75) | (77) | (65) | (66) | (71) | (72) |
| >6 | mm | n (%) | 21 | 24 | 30 | 23 | 23 | 34 | 23 | 30 | 208 |
| | | | (15) | (18) | (22) | (17) | (17) | (25) | (18) | (23) | (19) |

| | | | | | | | | | | |
|---|---|---|---|---|---|---|---|---|---|---|
| Target toe treatment | Mean | 1 | 1 | 1 | 1 | 1 | 1 | 1 | 1 | — |
| time (min) | | | | | | | | | | |
| Syringe used | n | 85 | 85 | 88 | 86 | 88 | 89 | 87 | 88 | 696 |
| Syringe failed | n | 0 | 0 | 0 | 0 | 1 | 0 | 0 | 0 | 1 |
| Cannula used | n | 205 | 204 | 203 | 202 | 199 | 200 | 197 | 195 | 1,605 |
| Cannula failed | n | 0 | 0 | 0 | 0 | 0 | 0 | 0 | 0 | 0 |

* Patient 01-06 missed V03; Patient 02-08 early terminated since V04; Patient 03-03 lost to follow up since V08
Denominator used in the percentage calculations: number of total cannulations each visit.

Table 3 shows the volume and amount of applied dose by visit

TABLE 3

| Visit | | V02 | V03 | V04 | V05 | V06 | V07 | V08 | V09 |
|---|---|---|---|---|---|---|---|---|---|
| # Patients/target toes | N | 47 | 46 | 46 | 46 | 46 | 46 | 45 | 45 |
| Volume of HSG 42 | Mean | 52.7 | 60.3 | 63.1 | 60.7 | 67.0 | 71.7 | 66.7 | 76.9 |
| applied to SUS (μl) | Median | 47.3 | 54.7 | 59.7 | 56.5 | 64.0 | 65.3 | 65.6 | 80.6 |
| | Mix, Max | 5.4, | 18.1, | 20.5, | 18.8, | 12.4, | 7.3, | 9.4, | 11.7, |
| | | 128.0 | 166.7 | 159.1 | 126.9 | 160.2 | 171.0 | 125.8 | 184.9 |
| Amount of terbinafine | Mean | 20.6 | 23.6 | 24.6 | 23.7 | 26.2 | 28.0 | 26.1 | 30.0 |
| applied to SUS (mg) | Median | 18.5 | 21.4 | 23.3 | 22.1 | 25.0 | 25.5 | 25.6 | 31.5 |
| | Mix, Max | 2.1, | 7.1, | 8.0, | 7.4, | 4.8, | 2.9, | 3.7, | 4.6, |
| | | 50.0 | 65.1 | 62.2 | 49.6 | 62.6 | 66.8 | 49.1 | 72.2 |
| Volume of HSG 42 | Mean | 130.2 | 140.2 | 139.1 | 142.5 | 148.5 | 158.7 | 155.0 | 163.2 |
| applied all toes (μl) | Median | 118.3 | 133.9 | 133.9 | 134.4 | 159.7 | 161.8 | 146.2 | 163.4 |
| | Mix, Max | 10.8, | 28.2, | 44.5, | 32.3, | 31.2, | 36.5, | 34.4, | 19.4, |
| | | 287.1 | 264.5 | 315.2 | 297.3 | 334.4 | 416.1 | 434.4 | 360.2 |
| Amount of terbinafine | Mean | 50.9 | 54.7 | 54.3 | 55.7 | 58.0 | 62.0 | 60.5 | 63.7 |
| applied all toes (mg) | Median | 46.2 | 52.3 | 52.3 | 52.5 | 62.4 | 63.2 | 57.1 | 63.8 |
| | Mix, Max | 4.2, | 11.0, | 17.4, | 12.6, | 12.2, | 14.2, | 13.4, | 7.6, |
| | | 112.1 | 103.3 | 123.1 | 116.1 | 130.6 | 162.5 | 169.7 | 140.7 |
| | ≥2-grade IGA | 25.10 | 24.56 | 27.27 | 26.86 | 25.95 | 26.52 | 26.79 | 35.65 |
| | improvement, | | | | | | | | |
| | Mean | | | | | | | | |
| | <2-grade IGA | 18.50 | 23.24 | 24.71 | 23.09 | 26.34 | 28.30 | 25.17 | 26.89 |
| | improvement, | | | | | | | | |
| | Mean | | | | | | | | |

* Patient 01-06 missed V03; Patient 02-08 early terminated since V04; Patient 03-03 lost to follow up since V08
Denominator used in the percentage calculations: number of target toes at each visit.

Dermatophytoma: Eight target toes (8/47, 17.0%) with visible, distinct dermatophytoma fungal masses were diagnosed by investigators at baseline and an outside blinded evaluator. The fungal masses were located in mid or lateral sections of the nail plate, and were patches or bands characterized by brown-yellow-white, even red discoloration. Of the eight patients enrolled with DLSO complicated by dermatophytoma (01-07, 02-03, 02-04, 02-07, 03-01, 03-11, 04-02, 04-06), four achieved Mycological Cure (4/8 50.0%), and all eight were culture negative (100%). The mean baseline disease-involved nail area of these particular patients was 52.41% and the mean disease-involved length was 78.17%. Of the eight target toes diagnosed with dermatophytoma, six patients assessed improvement (6/8, 75.0%); two patient assessed no change.

Plasma PK: Two designated PK sites (02, 04) drew blood from a subset of patients (N=15) for the determination of terbinafine and major metabolite N-desmethyl terbinafine concentrations in plasma. Eleven male and four female patients comprised the plasma PK subset. Blood was drawn day 1 (pre-dose), day 4, and pre-dose day 29, 57, and 85 (five time points). Characterization of human plasma concentrations of terbinafine and N-desmethyl terbinafine (ng/mL) over the study period was determined by bioanalytical lab KCAS using a validated LC/MS/MS method. The lower limit of quantification (LLOQ) for the bioanalytical method is 0.04-10.2 ng/ml for both parent and metabolite with a 0.150 mL aliquot volume. The total body doses of terbinafine administered to patients in the plasma PK subset of study HSG-201 are summarized in Table 4.

TABLE 4

| | Week 0, Day 1 | Week 4, Day 29 | Week 8, Day 57 |
|---|---|---|---|
| Mean (mg) | 51 | 53 | 51 |
| SD | 31 | 28 | 31 |
| Range (mg) | 18-106 | 11-103 | 17-123 |

No quantifiable terbinafine concentrations were measured in any pre-dose plasma samples collected on day 1. Of the seventy-four plasma samples obtained from fifteen patients, twenty-one samples (from 11 of 15 patients) contained quantifiable terbinafine concentrations. Of the twenty-one plasma samples with terbinafine concentrations reported above the LLOQ of 0.0400 ng/ml, ten were less than 1.5-fold greater than LLOQ (range: 0.0401 ng/ml to 0.0572 ng/ml). Terbinafine and N-DT plasma exposures were negligible after multiple doses. Of the eleven patients with quantifiable terbinafine plasma concentrations, Tmax occurred at the first post dose sampling time of week 0, day 4. In patient 04-01, Tmax occurred at day 57. The mean Cmax of 0.1293 ng/mL is 7700-fold less than the plasma Cmax derived from a single oral dose of 250 mg and 9700-fold less than the Cmax following multiple daily doses of Lamisil.

Nail Terbinafine Concentration: target toe distal nail clippings were collected from all patients enrolled in HSG-201 and analyzed for terbinafine and N-desmethyl terbinafine concentrations. Distal nail clippings were to be collected pre-treatment day 1 (week 0), week 4 day 29 (week 4), day 57 (week 8), day 85 (week 12, day 141 (week 20), day 197 (week 28), day 253 (week 36), day 309 (week 44), day 337 (week 48, and week 52 day 365 (week 52). The target toe terbinafine doses administered to patients enrolled in the nail PK subsets are summarized in Table 5.

TABLE 5

| | Mean (mg) | SD | Range (mg) |
|---|---|---|---|
| Week 0, Day 1 | 20.6 | 11.0 | 2.1-50.0 |
| Week 4, Day 29 | 23.6 | 12.0 | 7.1-65.1 |
| Week 8, Day 57 | 24.6 | 11.0 | 8.0-62.2 |
| Week 12, Day 85 | 23.7 | 10.1 | 7.4-49.6 |
| Week 20, Day 141 | 26.2 | 13.1 | 4.8-62.6 |
| Week 28, Day 197 | 28.0 | 15.7 | 2.9-66.8 |
| Week 36, Day 253 | 26.1 | 12.1 | 3.7-49.1 |
| Week 44, Day 309 | 30.0 | 16.0 | 4.6-72.2 |

Characterization of concentrations of terbinafine and N-desmethyl terbinafine in distal nail clippings (ug/g) was determined by KCAS using qualified LC/MS/MS methods. The bioanalytical methods were qualified using LC-MS/MS techniques to quantify terbinafine and N-DT in human nail hydrolysate; two methods were qualified due to the wide range of nail concentrations anticipated. The low-curve method was linear over the range of 0.500 to 64.0 ng/ml with a 0.100 mL aliquot volume for both terbinafine and N-DT. The high curve method was linear over the range of 50.0 to 100,000 ng/mL with a 0.100 mL aliquot volume for both terbinafine and N-DT. Both methods were approved for use on unknown human nail hydrolysate samples for non-GLP only.

Across all target toe sampling days, target toe pre-dose terbinafine maximal mean concentrations ranged from 161 ug/mg to 899 ug/mg and pre-dose N-DT maximal mean concentrations ranged from 0.663 ug/mg to 2.20 ug/mg. The across-all-dose-days mean terbinafine concentration of 99.54 ug/mg ranged from approximately 181,000-fold to 399,000-fold greater than terbinafine distal toenail clipping concentrations derived from oral terbinafine 250 mg once daily for up to 48 weeks.

Adverse Events—Study Related: Seven adverse events considered related to HSG 42 by the investigator were reported in six patients, including an event of mild burning or stinging in one patient (2.1%), three events of mild pain in three patients (6.4%), two events of moderate pain in two patients (4.3%) and one event of mild subungual hematoma in one patient (2.1%). Table 6 depicts the results for adverse events, and Table 7 lists the results at week 52 for all subjects.

TABLE 6

| | Number of Patients (%) | | | | |
|---|---|---|---|---|---|
| Adverse Events | Mild | Moderate | Severe | Any Grade | Toe |
| Burning/Stinging | 1 (2.1) | — | — | 1 (2.1) | TGT |
| Pain | 3 (6.4) | 2 (4.3) | — | 5 (10.6) | 4 TGTs; 1 non-TGT |
| Subungual | 1 (2.1) | — | — | 1 (2.1) | TGT |
| Hematoma | | | | | |

Denominator used in the percentage calculations: patients in safety population = 47

TABLE 7

| | | | | | | | | | | | | | | |
|---|---|---|---|---|---|---|---|---|---|---|---|---|---|---|
| | | | | | | | | Change | 80% | Change | 80% | | | Change |
| | | | | | | | | from | improver | from | iprover | | | from |
| | | | | Clini- | | | Myco- | base- | based | base- | based | | | base- |
| | Patient | | Complete | cal | | | logical | line - | on % | line - | on % | Positive | IGA | line - |
| Site | ID | TGT | cure | Cure | KOH | Culture | cure | % NPIA | NPIA | % NPIL | NPIL | Responder | V11 | IGA |

| | | | | | | | | | | | | | |
|---|---|---|---|---|---|---|---|---|---|---|---|---|---|
| 01 | 01-01 | R | No | No | Positive | Negative | No | 80.1% | Yes | No | Yes | 2 | 2 |
| 01 | 01-02 | L | No | No | Positive | Negative | No | 54.6% | No | No | Yes | 2 | 2 |
| 01 | 01-03 | L | No | No | Positive | Negative | No | 34.1% | No | No | Yes | 3 | 1 |
| 01 | 01-04 | R | No | No | Positive | Negative | No | 61.7% | No | No | Yes | 2 | 1 |
| 01 | 01-05 | R | Yes | Yes | Negative | Negative | Yes | 100.0% | Yes | Yes | Yes | 0 | 3 |
| 01 | 01-06 | R | Yes | Yes | Negative | Negative | Yes | 100.0% | Yes | Yes | Yes | 0 | 4 |
| 01 | 01-07 | L | No | No | Negative | Negative | Yes | 81.6% | Yes | No | Yes | 1 | 2 |
| 01 | 01-08 | R | No | Yes | Positive | Negative | No | 100.0% | Yes | Yes | Yes | 0 | 3 |
| 01 | 01-09 | L | No | No | Positive | Negative | No | 77.9% | No | No | Yes | 1 | 2 |
| 01 | 01-10 | R | No | No | Positive | Positive | No | 97.2% | Yes | No | Yes | 1 | 2 |
| 01 | 01-11 | L | No | Yes | Positive | Negative | No | 100.0% | Yes | Yes | Yes | 0 | 3 |
| 01 | 01-12 | L | No | No | Positive | Negative | No | 25.7% | No | No | Yes | 3 | 0 |
| 01 | 01-13 | L | No | Yes | Positive | Negative | No | 100.0% | Yes | Yes | Yes | 0 | 3 |
| 01 | 01-14 | R | No | No | Positive | Negative | No | 28.3% | No | No | Yes | 3 | 0 |
| 01 | 01-15 | R | No | No | Positive | Negative | No | 93.9% | Yes | No | Yes | 1 | 3 |
| 01 | 01-16 | L | No | No | Positive | Negative | No | 71.3% | No | No | Yes | 2 | 2 |
| 02 | 02-01 | L | No | No | Negative | Negative | Yes | −2.6% | No | No | Yes | 4 | 0 |
| 02 | 02-02 | L | No | No | Positive | Positive | No | −18.0% | No | No | Yes | 4 | −1 |
| 02 | 02-03 | L | No | No | Positive | Negative | No | −2.7% | No | No | Yes | 4 | 0 |
| 02 | 02-04 | R | No | No | Negative | Negative | Yes | 64.3% | No | No | Yes | 2 | 2 |
| 02 | 02-05 | L | No | No | Positive | Negative | No | 45.6%* | No | No | No | 2* | 1* |
| 02 | 02-06 | R | No | No | Positive | Negative | No | −9.3% | No | No | Yes | 4 | 0 |
| 02 | 02-07 | R | No | No | Positive | Negative | No | −13.0% | No | No | Yes | 4 | 0 |
| 02 | 02-08 | R | No | No | — | — | No | −3.9%* | No | No | No | 4* | 0* |
| 02 | 02-09 | R | No | No | Positive | Negative | No | 9.9% | No | No | Yes | 4 | 0 |
| 03 | 03-01 | R | No | No | Negative | Negative | Yes | 34.9% | No | No | Yes | 3 | 1 |
| 03 | 03-02 | L | No | No | Negative | Negative | Yes | 33.2% | No | No | Yes | 3 | 0 |
| 03 | 03-03 | L | No | No | — | — | No | 29.2%* | No | No | No | 3* | 1* |
| 03 | 03-04 | L | No | No | Positive | Negative | No | 22.9% | No | No | Yes | 3 | 1 |
| 03 | 03-05 | L | No | No | Negative | Negative | Yes | −10.9% | No | No | Yes | 4 | 0 |
| 03 | 03-06 | R | No | No | Positive | Negative | No | 9.1% | No | No | Yes | 3 | 0 |
| 03 | 03-07 | R | No | No | Positive | Negative | No | −123.6% | No | No | Yes | 4 | −1 |
| 03 | 03-08 | R | No | No | Negative | Negative | Yes | 58.4% | No | No | Yes | 3 | 1 |
| 03 | 03-09 | L | No | No | Positive | Negative | No | −53.3% | No | No | Yes | 4 | −1 |
| 03 | 03-10 | R | No | No | Positive | Positive | No | 1.9% | No | No | Yes | 4 | 0 |
| 03 | 03-11 | L | No | No | Positive | Negative | No | 25.7% | No | No | Yes | 4 | 0 |
| 03 | 03-12 | R | No | No | Positive | Negative | No | 9.6% | No | No | Yes | 3 | 0 |
| 03 | 03-13 | L | No | No | Positive | Negative | No | −2.0% | No | No | Yes | 4 | 0 |
| 03 | 03-14 | R | No | No | Positive | Negative | No | 15.1% | No | No | Yes | 3 | 1 |
| 03 | 03-15 | L | No | No | Positive | Negative | No | 89.2% | Yes | No | Yes | 1 | 2 |
| 03 | 03-16 | R | No | No | Positive | Negative | No | −26.0% | No | No | Yes | 4 | 0 |
| 04 | 04-01 | L | No | No | Positive | Negative | No | 15.3% | No | No | Yes | 3 | 0 |
| 04 | 04-02 | L | No | No | Positive | Negative | No | −1.8% | No | No | Yes | 3 | 0 |
| 04 | 04-03 | R | No | No | Positive | Negative | No | −53.2% | No | No | Yes | 4 | −1 |
| 04 | 04-04 | R | No | No | Negative | Negative | Yes | 98.0% | Yes | No | Yes | 1 | 3 |
| 04 | 04-05 | R | No | No | Positive | Negative | No | −10.4% | No | No | Yes | 4 | 0 |
| 04 | 04-06 | R | No | No | Negative | Negative | Yes | −20.7% | No | No | Yes | 3 | 0 |

*Last observation carried forward data

Although some patients reached study completion with a positive KOH, the majority had negative fungal cultures. This indicates that HSG 42 was fungicidal to dermatophytes, and that it requires a full outgrowth of the nail in 52-weeks before a KOH test would be negative (note the KOH test visualizes fungal elements but indirectly could show presence of dead cells)). This may be challenging in older patients with a long history of infection and slower growing nails. Moreover, it should also be appreciated that due to the high retention of the antifungal agent in the treatment space and advancement of a newly formed healthy nail plate, onycholysis can be reduced or even entirely reversed, thereby also preventing recurrence of infection.

Evaluation of this treatment regimen (a total of eight HSG 42 doses administered once a month or once every other month over 44-weeks) in a moderate to severely distal lateral subungual onychomycosis (DLSO) infected population of forty-seven adults (mean age 56, infection duration 13.8 years, target toe nail thickness of 2.2 mm, target toe involved area and length of 47.6% and 72.5%, including some patients clinically diagnosed with dermatophytoma) showed meaningful clinical response in a number of patients evidenced by the following 52-week outcomes:

Two patients (2/47, 4.3%) achieved complete cure as assessed by the investigators. Five patients (5/47, 10.6%) achieved complete cure as assessed by the outside blinded evaluator.

Eleven patients (11/45, 24.4%) achieved mycological cure. The negative KOH rate on completed patients was 24.4% (11/45). The negative culture rate on completed patients was 93.3% (42/45). All forty-seven patients enrolled into the study at week zero (baseline) with positive KOH and culture. To eliminate any residual terbinafine in the final mycology sample, an eight-week no treatment follow-up period preceded KOH and culture testing at week 52.

According to investigators assessment, four patients (8.5%) achieved almost complete cure (≤10% involved nail with negative mycology); ten patients (10/47, 21.3%) achieved IGA grade 0-1 (≤10% involvement) with negative culture; fourteen patients (14/47, 29.8%) achieved a 2-grade improvement in IGA with negative culture. Here, it should be noted that positive responders and 2-grade IGA improvers received a significantly higher baseline (week zero, visit 2) dose than all other patients in the study (25.1 mg vs 18.5 mg). This indicates that more doses may be required early in treatment to fully halt advancing fungi across the nail bed so that sustained growth of new clear nail can occur in more patients. Also, positive responders and 2-grade improvers had shorter duration of infection (11.7 vs 14.8 years); thinner nails (1.9 mm vs 2.3 mm); and less subungual hyperkeratosis (0.5 mm vs. 0.9 mm) than other patients. These prognostic factors appeared to be more important in influencing a positive outcome than age or percent nail involvement.

Of the fifteen 2-grade improvers assessed by investigators, two were complete cures (2/15, 13.2%); five were clinical cures (5/15, 33.3%), five were mycological cures (5/15, 33.3%), five were negative KOH (5/15, 33.3%), fourteen had negative culture (14/15, 93.3%), IGA improved by 2.5 grades, growth of uninvolved nail was a mean of 7.4 mm, total nail thickness reduced 0.4 mm (1.9 mm to 1.5 mm), nail only thickness reduced 0.4 mm (1.4 mm to 1.0 mm), and distal onycholysis (pre-existing) reduced 0.3 mm (0.5 mm to 0.2 mm).

Thirty-seven patients (37/45, 82.2%) saw improvement of DLSO in their target toe based on Patient Global Assessment; six of these patients (6/45, 13.3%) assessed completely clear; twelve (12/45, 26.7%) marked improvement; six (6/45, 13.3%) moderate improvement; thirteen (13/45, 28.9%) slight improvement; five (5/45, 11.1%) no change, and three (3/45, 6.7%) felt their target toe has worsened. Of the eight TGT's diagnosed with dermatophytoma, six patients assessed overall DLSO improvement (6/8, 75.0%); two patients assessed no change.

Overall, patients were compliant and most reported target toe improvement. Forty-two patients (42/46, 91%) assessed improvement of DLSO at week 20; however, this dropped to thirty-seven (37/45, 32.5%) at week 52 indicating that some patients experienced disease regression in the later stages of the study. Forty-five patients (45/47, 95.7%) completed the study. Only one patient missed a scheduled treatment.

### Refractory Dermatophytoma Treatment Example

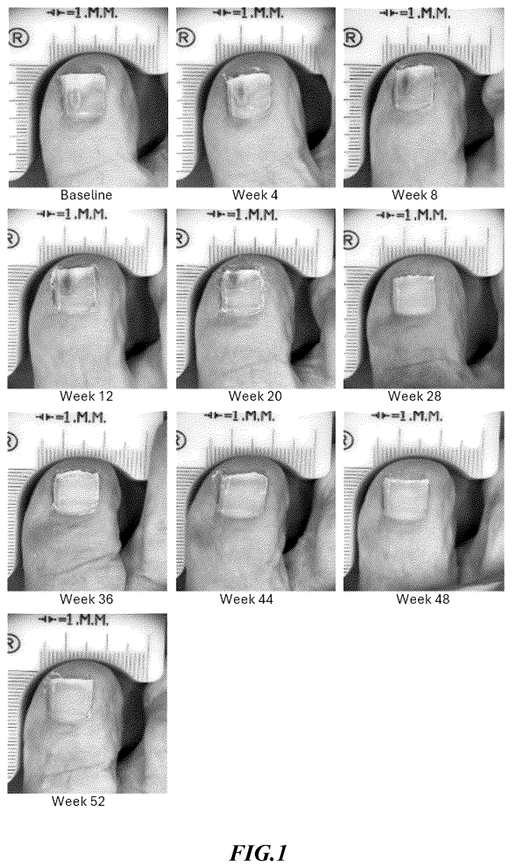

In a difficult-to-treat dermatophytoma case, a 56-year-old, active adult presented positive for T. rubrum culture with dermatophytoma of the great toe. Observed clinical signs of the infection included a distinct orange, brown band of dermatophytoma extending midline to the lunula. The nail was 1.4 mm thick, including 0.3 mm of subungual hyperkeratosis. Planimetry quantified 51% nail area involvement. The to-be-treated target toe is shown in FIG. 1 at ‘Baseline’. Treatment comprised 8 doses of high-concentration terbinafine as shown in F8 of Table 1 over 44 weeks. Bypassing the plate, the nail bed was treated directly using subungual space pathways accessed at the free edge caused by onycholysis. At week 20, involvement dropped to 31%, and 14% by week 36. At week 52, the nail was assessed as clinically cured by the outside blinded evaluator (0% disease-involved nail area). Mycology was negative (KOH and culture). Healthy nail had grown 6.6 mm and nail thickness had reduced 0.5 mm. Each application lasted 3 minutes. Note that over the course of treatment the administered quantity of the formulation decreased, which is indicative of a decreased subungual space, which in turn is a reflection of reattachment of healthy nail plate to a healthy nail bed. As such, treatment was also shown to reduce onycholysis.

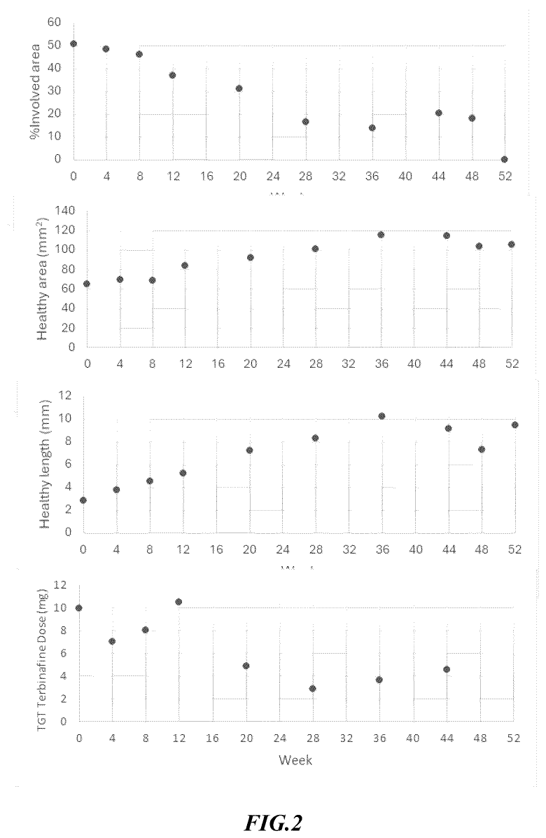

Treatment progress is shown in the remaining photographs in FIG. 1, and FIG. 2 depicts exemplary metric for treatment progress as indicated. At week 52, the dermatophytoma resolved and onychomycosis cleared. The patient assessed the nail as disease cleared. Such treatment success is particularly notable as each application lasted only 3 mins at point-of-care without pain or local site reactions during or after treatment. No anesthetic was required, and no systemic safety concerns were observed.

While for the above examples the composition F8 of Table 1 above was used, it should be appreciated that numerous alternate compositions will also be suitable in conjunction with the teachings herein, and especially contemplated alternate compositions are described above and in U.S. Pat. No. 11,400,061, incorporated by reference herein.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method of treating dermatophytoma, comprising:
subungually and atraumatically administering a composition proximal to and/or into a polysaccharide matrix of a dermatophytoma located in a subungual space;
wherein the composition has a viscosity of between about 500-2,500 cP (mPa*s) and comprises an antifungal agent in a hydrophobic solvent;
wherein the antifungal agent has at a concentration effective to inhibit growth of a fungal pathogen in the subungual space and to inhibit growth of the fungal pathogen in the nail plate as ascertained by a negative culture of distal nail clippings; and
wherein the composition is administered repeatedly to so treat the dermatophytoma.

2. The method of claim 1, wherein the hydrophobic solvent is selected from the group consisting of isostearic acid, benzyl alcohol, diisopropyl adipate, diethyl sebacate, isopropyl myristate, and combinations thereof.

3. The method of claim 1, wherein the composition further comprises a hydrophilic solvent and/or a polymeric film forming agent.

4. The method of claim 1, wherein the antifungal agent is terbinafine free base.

5. The method of claim 1, wherein the antifungal agent is present at a concentration that sustains an antifungal drug concentration of at least 1× minimum inhibitory concentration (MIC) for at least 30 days.

6. The method of claim 1, wherein the composition is formulated to substantially completely prevent systemic absorption of the antifungal agent into blood circulation.

7. The method of claim 1, wherein the composition is administered monthly for at least one year.

8. A method of treating onychomycosis, comprising:
subungually and atraumatically administering a composition into a subungual space at an asymmetric schedule;
wherein the composition comprises a pharmaceutically acceptable carrier comprising an antifungal agent in a hydrophobic solvent, an optional hydrophilic solvent, an optional polymeric film forming agent, wherein the composition has a viscosity of between about 500-2,500 cP (mPa*s);
wherein the asymmetric schedule comprises a loading schedule that extends over at least two weeks and a maintenance schedule that extends over at least six month; and
wherein the loading schedule delivers, normalized per month, at least twice an amount of the antifungal agent to the subungual space relative to the maintenance schedule.

9. The method of claim 8, wherein the hydrophobic solvent is selected from the group consisting of isostearic acid, benzyl alcohol, diisopropyl adipate, diethyl sebacate, isopropyl myristate, and combinations thereof, and wherein the composition further comprises the hydrophilic solvent and/or the polymeric film forming agent.

10. The method of claim 8, wherein the antifungal agent is terbinafine free base at a concentration of at least 10 wt %.

11. The method of claim 8, wherein the asymmetric schedule extends over one year.

12. The method of claim 8, wherein the loading schedule extends over one month and comprises one initial and two biweekly administrations, and wherein the maintenance schedule extends over one year and comprises eleven once-monthly administrations.

13. The method of claim 8, wherein an Investigator Global Assessment (IGA) grade improves at least 2 grades after completion of the asymmetric schedule.

14. The method of claim 8, wherein, upon completion, the asymmetric schedule is capable of producing complete cure and/or mycological cure.

15. A method of treating onychomycosis, comprising:
subungually and atraumatically administering a composition into a subungual space at a metronomic schedule;
wherein the composition comprises a pharmaceutically acceptable carrier comprising an antifungal agent in a hydrophobic solvent, an optional hydrophilic solvent, an optional polymeric film forming agent, wherein the composition has a viscosity of between about 500-2,500 cP (mPa*s);
wherein the composition is formulated such that, upon completion of the metronomic schedule, (1) growth of the fungal pathogen in the nail plate is inhibited as ascertained by a negative culture of distal nail clippings, (2) antifungal agent is present in the nail plate, and (3) antifungal agent is substantially absent in systemic circulation.

16. The method of claim 15, wherein the hydrophobic solvent is selected from the group consisting of isostearic acid, benzyl alcohol, diisopropyl adipate, diethyl sebacate, isopropyl myristate, and combinations thereof, and wherein the composition further comprises the hydrophilic solvent and/or the polymeric film forming agent.

17. The method of claim 15, wherein the antifungal agent is terbinafine free base at a concentration of at least 10 wt %.

18. The method of claim 15, wherein a mean Cₘₐₓ of the antifungal agent is equal or less than 0.13 ng/mL in plasma post administration.

19. The method of claim 15, wherein a mean concentration of the antifungal agent in nail clippings is about 100 μg/mg.

20. The method of claim 15, wherein the metronomic schedule comprises a loading schedule and a maintenance schedule.
